(19) United States
(12) Patent Application Publication
(10) Pub. No.: US 2015/0005860 A1
(43) Pub. Date: Jan. 1, 2015

(54) PADDLE LEADS AND LEAD ARRANGEMENTS FOR DORSAL HORN STIMULATION AND METHODS AND SYSTEMS USING THE LEADS

(71) Applicant: BOSTON SCIENTIFIC NEUROMODULATION CORPORATION, Valencia, CA (US)

(72) Inventors: Joshua Dale Howard, Chatsworth, CA (US); Rafael Carbunaru, Valley Village, CA (US); Anne Margaret Pianca, Santa Monica, CA (US); Dongchul Lee, Agua Dulce, CA (US)

(21) Appl. No.: 14/311,983

(22) Filed: Jun. 23, 2014

Related U.S. Application Data
(60) Provisional application No. 61/840,240, filed on Jun. 27, 2013.

(51) Int. Cl. A61N 1/05

(52) U.S. Cl. CPC A61N 1/0553
USPC 607/117

(57) ABSTRACT

A spinal cord stimulation lead for dorsal born stimulation includes a paddle body having a distal end, a proximal end, and a longitudinal length extending from the distal end to the proximal end; at least one lead body having a distal end portion, a proximal end portion, and a longitudinal length, the distal end portion of each of the at least one lead body being coupled to the proximal end of the paddle body; electrodes disposed on the paddle body, where the electrodes from at least two longitudinal columns including a first column and a second column with the first and second columns spaced apart laterally by at least 7 mm, center-to-center; terminals disposed along the proximal end portion of the at least one lead body; and conductors electrically coupling the terminals to the electrodes.

### CROSS-REFERENCE TO RELATED APPLICATIONS

This application claims the benefit under 35 U.S.C. §119(e) of U.S. Provisional Patent Application Ser. No. 61/840,240, filed Jun. 27, 2013, which is incorporated herein by reference.

### FIELD

The present invention is directed to the area of implantable electrical stimulation systems and methods of making and using the systems. The present invention is also directed implantable electrical stimulation leads with electrodes for dorsal horn stimulation, as well as methods of making and using the leads and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissues to be stimulated. The pulse generator in the control module generates electrical pulses that are delivered by the electrodes to body tissue.

### BRIEF SUMMARY

One embodiment is a spinal cord stimulation lead including a paddle body configured and arranged to be implanted into the patient to stimulate a portion of the spinal cord of the patient, the paddle body having a distal end, a proximal end, and a longitudinal length extending from the distal end to the proximal end; at least one lead body having a distal end portion, a proximal end portion, and a longitudinal length, the distal end portion of each of the at least one lead body being coupled to the proximal end of the paddle body; electrodes disposed on the paddle body, wherein the electrodes form at least two longitudinal columns including a first column and a second column, wherein the first and second columns are spaced apart laterally by at least 7 mm, center-to-center; terminals disposed along the proximal end portion of the at least one lead body; and conductors electrically coupling the terminals to the electrodes.

Another embodiment is a spinal cord stimulation system including the spinal cord stimulation lead describe above and a control module coupleable to the spinal cord stimulation lead.

Yet another embodiment is a method of spinal cord stimulation. The method cord stimulation lead describe above and a control module-coupleable to the spinal cord stimulation lead.

Yet another embodiment is a method of spinal cord stimulation. The method includes implanting at least one electrical stimulation lead over a spinal cord of a patient so that a first column of electrodes is disposed over a first dorsal born of the spinal cord and a second column of electrodes is disposed over a second dorsal born of the spinal cord that is opposite the first dorsal horn; coupling the at least one electrical stimulation lead to a control module: and applying a stimulation current generated by the control module to at least one of the first or second dorsal horns using at least one of the electrodes of the first or second columns of electrodes.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understood of the present invention, reference will be made to the following Detailed Description, which is to be read in association with the accompanying drawings, wherein:

FIG. 1 is a schematic view of one embodiment of an electrical stimulation system that includes a lead electrically coupled to a control module, according to the invention;

FIG. 2A is a schematic view of one embodiment of the control module of FIG. 1 configured and arranged to electrically couple to an elongated device, according to the invention;

FIG. 2B is a schematic view of one embodiment of a lead extension configured and arranged to electrically couple the elongated device of FIG. 2A to the control module of FIG. 1, according to the invention;

FIG. 3 is a schematic cross-sectional view of a portion of a spinal cord;

FIG. 4 is a schematic top view of one embodiment of a paddle for a paddle lead, according to the invention;

FIG. 5 is a schematic top view of a second embodiment of a paddle for a paddle lead, according to the invention;

FIG. 6 is a schematic top view or a third embodiment of a paddle for a paddle lead, according to the invention;

FIG. 7 is a schematic top view of a fourth embodiment of a paddle for a paddle lead, according to the invention;

FIG. 8 is a schematic top view of a fifth embodiment of a paddle for a paddle lead, according to the invention;

FIG. 9 is a schematic top view of a sixth embodiment of a paddle for a paddle lead, according to the invention;

FIG. 10 is a schematic top view of one embodiment of an arrangement of the distal ends of four cylindrical lead bodies for electrical stimulation, according to the invention;

FIG. 11 is a schematic top view of a second embodiment of an arrangement of the distal ends of four cylindrical lead bodies for electrical stimulation, according to the invention;

FIG. 12 is a schematic overview of one embodiment of components of a stimulation systems, including an electronic subassembly disposed within a control module, according to the invention.

### DETAILED DESCRIPTION

The present invention is directed to the area of implantable electrical stimulation systems and methods of making and using the systems. The present invention is also directed implantable electrical stimulation leads with electrodes for dorsal horn stimulation, as well as methods of making and using the leads and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads and paddle leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 8,175,710; 8,224,450; 8,271,094; 8,295,944; 8,364,278; and 8,391,985; U.S. Patent Applications Publications Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615, all of which are incorporated by reference.

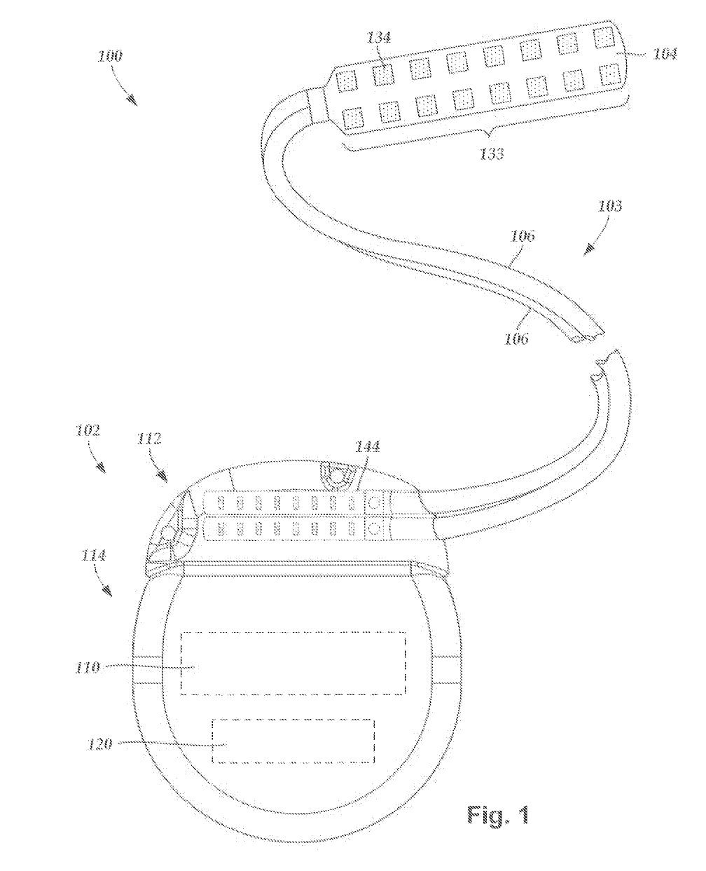

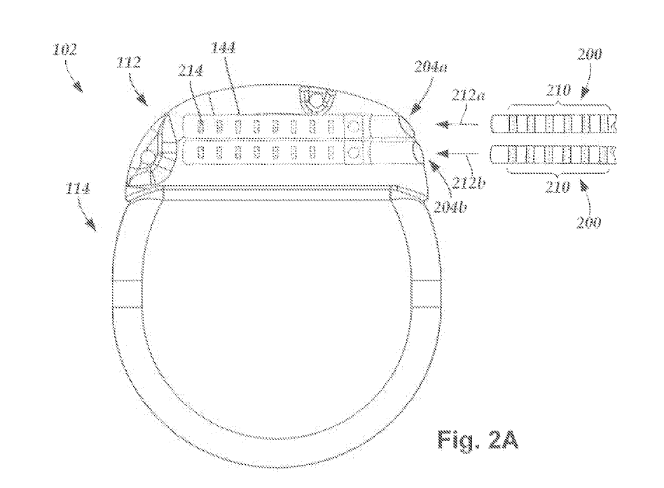

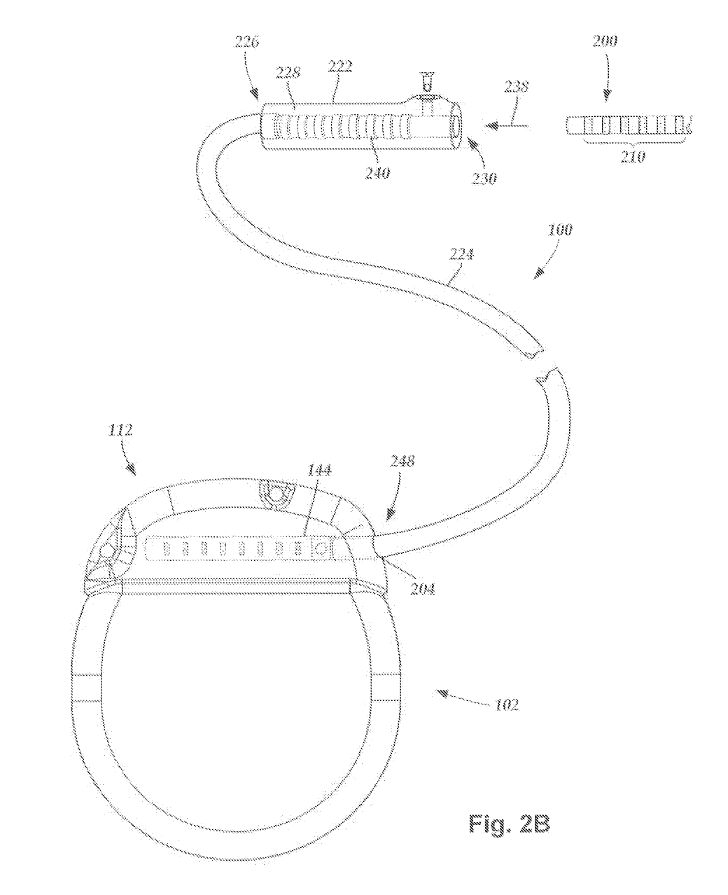

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two three, four, five, six, seven, eight or more lead bodies 106. An array of electrodes 133, such as electrodes 134, is disposed on the paddle body 104, and an array of terminals (e.g., 210 in FIG. 2A-2B) is disposed along each of the one or more lead bodies 106,

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices. For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

The control module 102 typically includes a connector housing 112 and a sealed electronics housing 113. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the paddle body including, for example, four, five, six, seven, eight, nine, ten, eleven, twelve, fourteen, sixteen, twenty-four, thirty-two, or more electrodes 134. The electrodes 134 can be disposed on the paddle body 104 in any suitable arrangement. In FIG. 1, the electrodes 134 are arranged into two columns, where each column has eight electrodes 134.

The electrodes of the paddle body 104 are typically disposed in, or separated by, a non-conductive, biocompatible material such as, for example, silicone, polyurethane, polyetheretherketone (“PEEK”), epoxy, and the like or combinations thereof. The paddle body 104 and the one or more lead bodies 106 may be formed in the desired shape by any process including, for example, molding (including injection molding), casting, and the like. The non-conductive material typically extends from the paddle body 104 to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachable coupled together.

Terminals (e.g., 210 in FIGS. 2A-2B) are typically disposed along the proximal end of the one or more lead bodies 106 of the electrical stimulation system 100 (as well as any splitters, lead extensions, adaptors, or the like) for electrical connection to corresponding connector contacts (e.g., 214 in FIGS. 2A-2B). The connector contacts are disposed in connectors (e.g., 144 in FIGS. 1-2B; and 222 FIG. 2B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably scalable at the distal end.

FIG. 2A is a schematic side view of one embodiment of a proximal end of one or more elongated devices 200 configured and arranged for coupling to one embodiment of the control module connector 144. The one or more elongated devices may include, for example, one or more of the lead bodies 106 of FIG. 1, one or more intermediate devices (e.g., a splitter, the lead extension 224 of FIG. 2B, an adaptor, or the like or combinations thereof), or a combination thereof.

The control module connector 144 defines at least one port into which a proximal end of the elongated device 200 can be inserted, as shown by directional arrows 212a and 212b. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 2B is a schematic side view of another embodiment of the electrical stimulation system 100. The electrical stimulation system 100 includes a lead extension 224 that is configured and arranged to couple one or more elongated devices 200 (e.g., one of the lead bodies 106 of FIG. 1, a splitter, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144 (e.g., the ports 204a and 204b of FIG. 1), or to receive multiple elongated devices 200 (e.g., both of the lead bodies 106 of FIG. 1), or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the bad extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

Stimulation of patient tissue, such as the spinal cord, can be useful in reducing pain and providing other therapy. It has been found that utilizing poise frequencies that are higher than conventional stimulation devices or pulse widths that are lower than conventional devices, which typically stimulate the dorsal column, can produce stimulation in the dorsal horn region while often lowering the undesirable side-effect of paresthesia. The dorsal horn can be stimulated using relatively low pulse width (for example, 30 μs or less.) Convention spinal cord stimulation systems typically use pulse widths of 200 μs or greater. It is believed that low pulse width allows for recruitment of nerve terminals or synapses in the dorsal horn of the spinal cord which may result in the modulation of pain transmission without orthodromic conduction of action potentials to the brain which can result in paresthesia. It is thought this occurs because the terminals in the dorsal horn of the spinal cord are very sensitive to low pulse width and because it is more difficult for action potentials generated in smaller fibers to elicit action potentials in post-synaptic fibers of larger diameter.

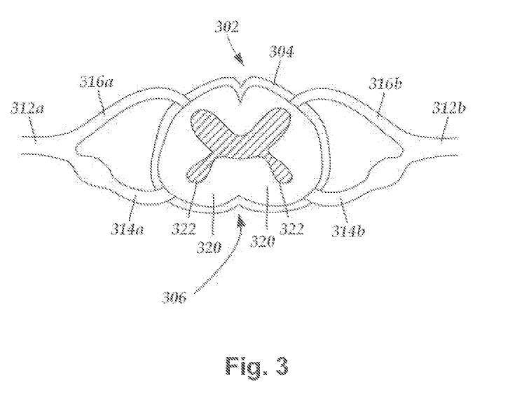

FIG. 3 schematically illustrates a transverse cross-sectional view of a spinal cord 302 surrounded by dura 304. The spinal cord 302 includes a midline 306 and a plurality of levels from which spinal nerves 312a and 312b extend. In FIG. 3A, the spinal nerves 312a and 312b are shown attaching to the spinal cord 302 at a particular spinal cord level via corresponding dorsal roots 314a and 314b and ventral (or anterior) roots 316a and 316b. Typically, the dorsal roots 314a and 314b relay sensory information into the spinal cord 302 and the central roots 316a and 316b relay motor information outward from the spinal cord 302.

The spinal cord 320, as illustrated in FIG. 3, also includes the dorsal (or posterior) column 320 and the dorsal (or posterior) horns 322. Conventional paddle leads have electrodes arranged to stimulate the dorsal column. In contrast to conventional leads, paddle leads can be designed to stimulate the dorsal horn in addition to, or as an alternative to, stimulation of the dorsal column. These paddles leads include columns of electrodes along the paddle that spaced further apart laterally than electrodes used for dorsal column stimulation because the dorsal horns are outside the dorsal column. For example, to stimulate the dorsal horn the columns of electrodes can be spaced apart laterally by 7 to 12 mm (center-to-center) or more. Many paddle leads have a lateral width of no more than 8 mm of less and, therefore, center-to-center lateral spacing between two columns of electrodes is in the range of 6 mm or less.

Each of the leads described below includes a paddle with two columns of electrodes that are arranged to stimulate the two dorsal horns of the spinal cord. In at least some embodiments, each of the leads described below has the two columns arranged with a lateral separation (center-to-center distance) of at least 7, 8, 9, 10, 11, or 12 mm or more. In at least some embodiments, each of the leads described below has the two columns arranged with lateral separation (center-to-center distance) that is at least equal to an average separation distance between the ends of the dorsal horns of an average adult human (or adult human male or adult human female) in one of the thoracic region, the lumbar region, or the cervical region (or for any particular vertebral level (or range of adjacent levels, for example, T8-T10) within any of those regions) of the spinal cord. As described below, the paddle may also include other electrodes that are arranged to stimulate the dorsal column of the patient.

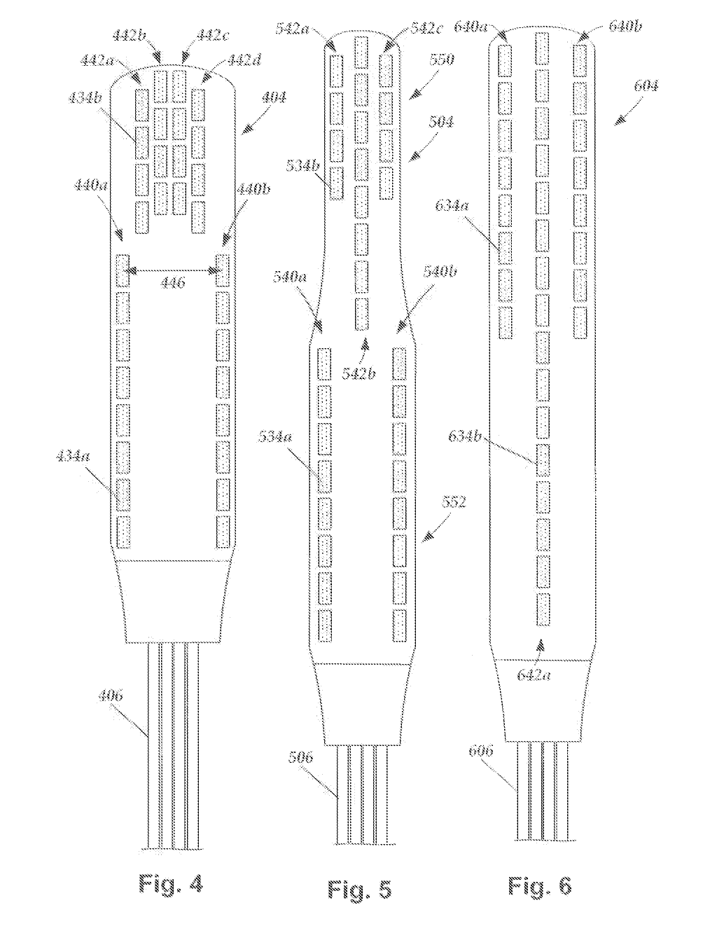

With respect to the embodiments illustrated in FIGS. 4-9, the reference numerals of equivalent structures in those embodiments differ only in the first digit. For example, reference numerals 404, 504, 604, 704, 804, an 904 all refer to a paddle. It will be understood that, unless indicated otherwise, any discussion below with respect to a structure of a particular embodiment is also applicable to the equivalent structures in the other embodiments. For example, any description related to electrodes 434a is also applicable, unless indicated otherwise, to electrodes 534a, 634a, 734a, 834a, and 934a.

A paddle lead can include electrodes that are situated on the paddle to provide dorsal horn stimulation and other electrodes that are situated on the paddle to provide dorsal column stimulation. FIG. 4 illustrates a paddle 404 of a paddle lead with one or more lead bodies 406 extending from the paddle. The paddle 404 includes columns 440a, 440b, of electrodes 434a that are situated on the paddle so that they can be used to stimulated the dorsal horns of the spinal cord when the paddle lead is implanted near the spinal cord (for example, in the epidural space.) The center-to-center separation between the columns 440a, 440b, is indicated by line 446.

Each of the columns 440a, 440b can include any number of electrodes 434a including, nut not limited to, one, two, three, four, five, six, seven, eight, nine, ten, twelve, sixteen, or more electrodes. The electrodes 434a in each of the columns 440a, 440b can be spaced apart longitudinally in a uniform manner, as illustrated in FIG. 4, or in any other regular or irregular pattern. The electrodes 434a can be identical in size and shape or differ in size or shape. The columns 440a, 440b may have the same number or electrodes 434a or different numbers of electrodes. The columns 440a, 440b can be identical with respect to arrangement of the electrodes 434a or can be different. The electrodes of one column can be aligned with the electrodes of the other column or they can be unaligned or any combination thereof.

The paddle 404 also includes columns 442a, 442b, 442c, 442d of electrodes 434b that are arranged on the paddle so that they can be used to stimulate the dorsal column. The center-to-center lateral spacing between adjacent columns for this arrangement is generally no more than 6, 5, 4, 3, or 2 mm and may be in the range of 1 to 5 mm. The entire arrangement generally has a center-to-center lateral spacing between the two outermost columns 442a, 442d of no more than 6 mm and may be in the range of 1 to 5 mm.

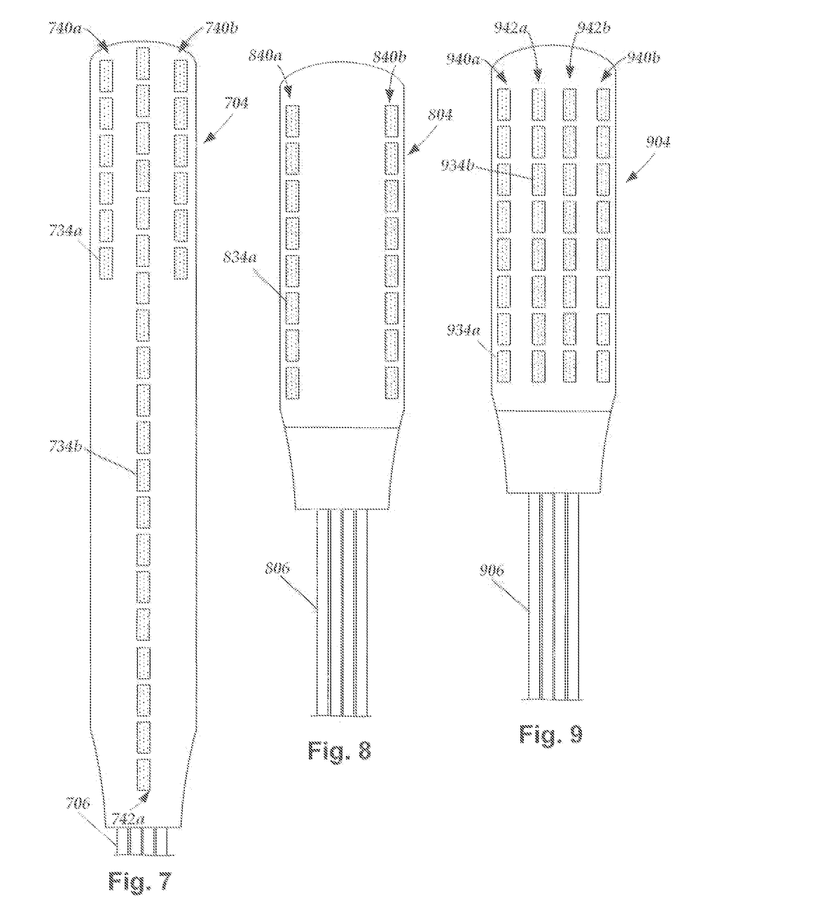

The paddle 404 is illustrated as having four columns 442a, 442b, 442c, 442d for stimulation the dorsal column, but it will be understood that such arrangement can have any number of columns including, but not limited to, one (see, e.g., FIGS. 6 and 7), two (see, e.g., FIG. 9), three (see, e.g., FIG. 5), four, six, or more columns. Each of the columns 442a, 442b, 442c, 442d can include any number of electrodes 434b including, but not limited to, one, two, three, four, five, six, seven, eight, nine, ten, twelve, sixteen, or more electrodes. In at least some embodiments, one of the columns is disposed on the midline of the paddle (see, e.g., FIG. 5, 6, and 7).

The electrodes 434b in each of the columns 442a, 442b, 442c, 442d can be spaced apart longitudinally in a uniform manner, as illustrated in FIG. 4, or in any other regular or irregular pattern. The electrodes 434b can be identical in size and shape or differ in size or shape. The columns 442a, 442b, 442c, 442d may have the same number of electrodes 434b or different numbers of electrodes. The columns 442a, 442b, 442c, 442d can be identical with respect to arrangement of the electrodes 434b or can be different. The electrodes of one column can be aligned with the electrodes of any other column(s) or they can be unaligned or any combination thereof. For example, in FIG. 4 the electrodes of column 442a are aligned with the electrodes of column 442d and the electrodes of column 442b are aligned with the electrodes of column 442c, but the electrodes of column 442a are not aligned with the electrodes of columns 442b and 442c.

The columns (e.g., columns 440a, 440b) of electrodes for dorsal horn stimulation can be longitudinally separated from the columns (e.g., columns 442a, 442b, 442c, 442d) of electrodes for dorsal column stimulation, as illustrated in FIGS. 4 and 5, or the two sets of columns can longitudinally overlap where the overlap is partial, as illustrated in FIGS. 6 and 7, or full, as illustrated in FIG. 9. If there is longitudinal separation between the two sets of column, in at least some embodiments, the separation distance is in the range of 1to 6 mm and may be at least 1, 2, 3, 4, or 5 mm. If the two sets of columns overlap, the electrodes of the columns of electrodes for dorsal horn stimulation can be aligned in the overlapping region with the electrodes of the columns of electrodes for dorsal column stimulation (see, FIG. 9) or not aligned (see, FIGS. 6 and 7).

The paddle can be uniform in width (except at the proximal and distal ends) as illustrated in FIGS. 4 and 6-9. In other embodiments, the paddle may be non-uniform in width, as illustrated in FIG. 5. For example, the paddle can be narrower in the region 550 where the electrodes for dorsal column stimulation reside and wider in the region 552 where the electrodes for dorsal horn stimulation reside. This non-uniform paddle width can be applied to, for example, the paddle configurations of FIGS. 4 and 6.

FIG. 5 illustrates a paddle 504 of a paddle lead with one or more lead bodies 506 extending from the paddle. The paddle 504 includes columns 540a, 540b, of electrodes 534a that are situated on the paddle to stimulate the dorsal horns of the spinal cord when the paddle lead is implanted near the spinal cord (for example, in the epidural space.) The paddle 504 also includes columns 542a, 542b, 542c, of electrodes 534b that are arranged on the paddle to stimulate the dorsal column. Column 542b has more electrodes 534b than columns 542a, 542c. When the paddle 504 is implanted, column 542b may be implanted over the midline of the spinal cord and the electrodes 534b of column 542b may be used, for example, to at least partially replicate stimulation that can be achieved along the midline using a conventional isodiametric percutaneous lead.

FIG. 6 illustrates a paddle 604 of a paddle lead with one or more lead bodies 606 extending from the paddle. The paddle 604 includes columns 640a, 640b of electrodes 634a that are situated on the paddle to stimulate the dorsal horns of the spinal cord when the paddle lead is implanted near the spinal cord (for example, in the epidural space.) There are eight electrodes 634a in each column 640a, 640b. The paddle 640 also includes a column 642a of sixteen electrodes 634b that are arranged on the paddle to stimulate the dorsal column. Column 642a has more electrodes than columns 640a, 640b. When the paddle 640 is implanted, column 642a may be implanted over the midline of the spinal cord and the electrodes 634b of column 642a may be used, for example, to at least partially replicate stimulation that can be achieved along the midline using a conventional isodiametric percutaneous lead.

FIG. 7 illustrates a paddle 704 of a paddle lead with one or more lead bodies 706 extending from the paddle. The paddle 704 includes columns 740a, 740b of electrodes 734a that are situated on the paddle to stimulate the dorsal horns of the spinal cord when the paddle lead is implanted near the spinal cord (for example, in the epidural space.) There are six electrodes 734a in each column 740a, 740b. The paddle 704 also includes column 742a of twenty electrodes 734b that are arranged on the paddle to stimulate the dorsal column. Column 742a has electrodes than columns 740a, 740. When the paddle 704 is implanted, column 742a may be implanted over the midline of the spinal cord and the electrodes 734b of column 742a may be used, for example, to at least partially replicate stimulation that can be achieved along the midline using a conventional isodiametric percutaneous lead.

In some embodiments, the paddle does not include electrodes specifically arranged for dorsal column stimulation (although stimulation of the dorsal horns may also result in some stimulation of the dorsal column.) FIG. 8 illustrates a paddle 804 of a paddle lead with one or more lead bodies 806 extending from the paddle. The paddle 804 includes columns 840a, 840b of electrodes 834a that are situated on the paddle to stimulate the dorsal horns of the spinal cord when the paddle lead is implanted near the spinal cord (for example, in the epidural space.) In at least some embodiments, a portion of the paddle 804 between the two columns 840a, 840b (such as the central portion of the paddle extending longitudinally along the length of the paddle) may be made substantially more flexible, particularly along the lateral direction, than other portions of the paddle so that the paddle can more easily bend around the roughly cylindrical shape of the spinal cord. The portion of the paddle may be made flexible by, for example, using different material(s) for the portion, or using the same general material (e.g., polyurethane or silicone) with a lower durometer or higher flexibility, or making the paddle thinner in the region. It will be understood that a similar flexible portion of the paddle can be incorporated into any of the other paddle described herein.

FIG. 9 illustrates a paddle 904 of a paddle lead with one or more lead bodies 906 extending from the paddle. The paddle 904 includes columns 940a, 940b of electrodes 934a that are situated on the paddle to stimulate the dorsal horns of the spinal cord when the paddle lead is implanted near the spinal cord (for example, in the epidural space.) The paddle 904 also includes columns 942a, 942b of electrodes 934b that are arranged on the paddle to stimulate the dorsal column.

Any of the paddle leads discloses herein can be implanted near the spinal cord (for example, in the epidural space) and coupled to a control module to provide stimulation cord through the electrodes to one or both dorsal horns or the dorsal column, or any combination thereof.

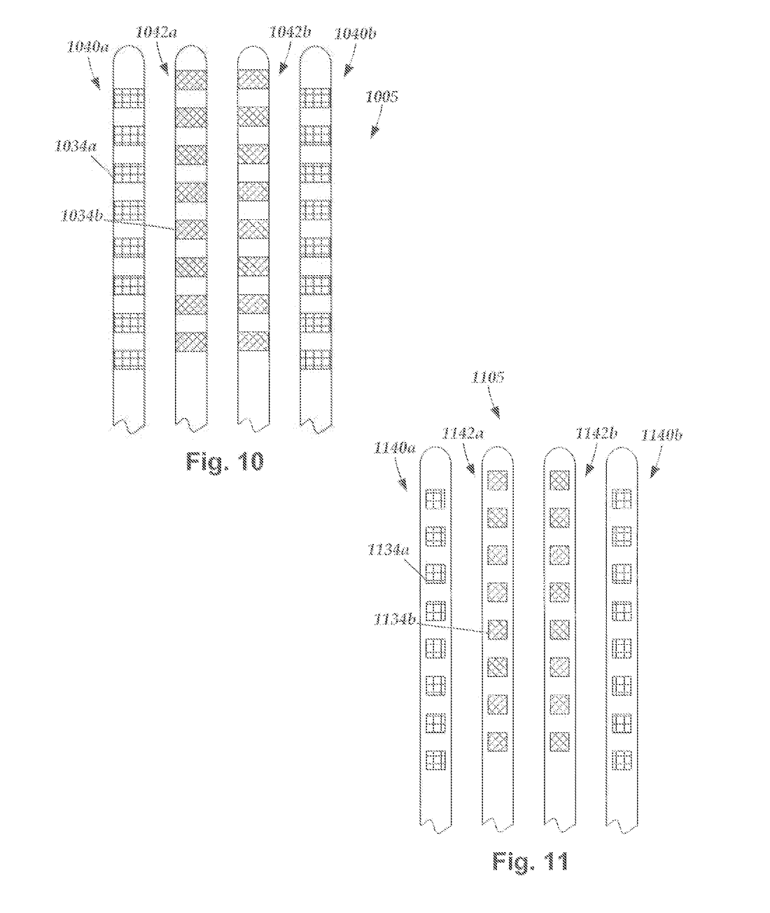

Alternatively, cylindrical lead bodies, such as those from percutaneous leads, can be used instead of a paddle. FIG. 10 illustrates four cylindrical lead bodies 1040a, 1042a, 1042b, and 1040b that are disposed in an arrangement similar to the arrangement of paddle 904 of FIG. 9. Lead bodies 1040a, 1040b include ring electrodes 1034a and are implanted to stimulate the dorsal horns. Lead bodies 1042a, 1042b include ring electrodes 1034b and are implanted to stimulate the dorsal column. The lead bodies 1040a, 1042a, 1042b, and 1040b can all be the distal ends of individual percutaneous leads or two or more of the lead bodies can be coupled together proximal to the portions illustrated in FIG. 10. Further description of percutaneous leads with single or multiple lead bodies can be found at, for example, U.S. Pat. No. 8,332,049 and U.S. Patent Application Publications Nos. 2010/0070009; 2011/0009933; 2011/0029052; 2012/0215295; and 2012/0316610, all of which are incorporated herein by reference.

FIG. 11 illustrates a similar arrangement of four cylindrical lead bodies 1140a, 1142a, 1142b, and 1140b. The electrodes 1034a, 1034b on these lead bodies are segmented electrodes. Each segmented electrode extends only part way (e.g., no more than 75%, 67%, 60%, 50%, 40%, 33%, 25%, 20%, 17%, or 15% or less) around the circumference of the lead body. In some embodiments, there may be multiple segmented electrodes disposed around the circumference of the lead at each longitudinal position. Further description of segmented electrodes can be found at, for example, U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321, all of which are incorporated herein by reference. It will also be understood that the lead bodies may incorporate any combination of ring electrodes and segmented electrodes and may also incorporate at tip electrode at the end of the lead.

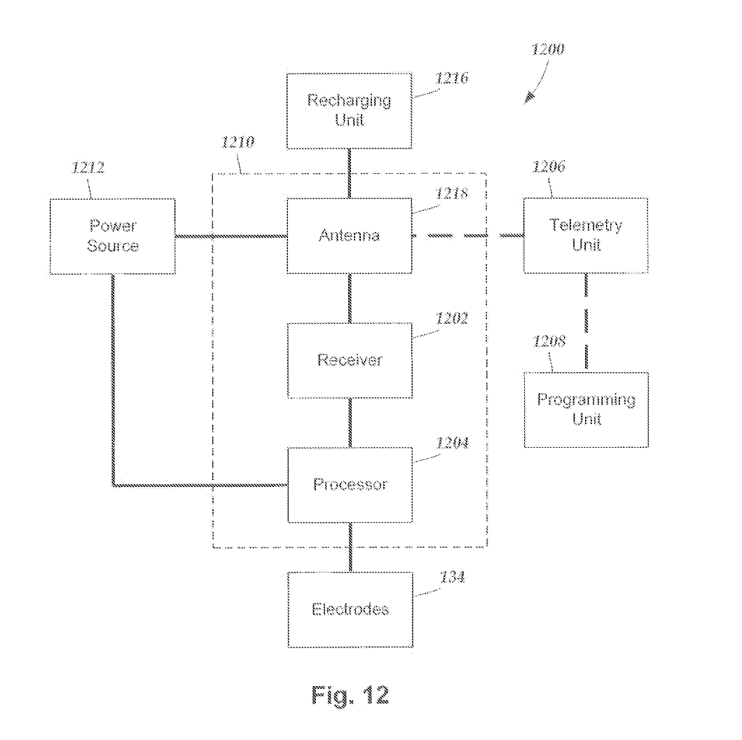

FIG. 12 is a schematic overview of one embodiment of components of an electrical stimulation system 1200 including an electronic subassembly 1210 disposed within a control module. It will be understood that the electrical stimulation system can include more, fewer, or different components and can have a variety of different configurations including those configurations disclosed in the stimulator references cited herein.

Although FIGS. 11 and 12 both illustrate using four lead bodies, it will be understood that other embodiments will include two lead bodies for distal horn stimulation and any number of lead bodies (including, but not limited to, zero, one, two, three, four, six, or more lead bodies) for dorsal column stimulation. The electrodes of the lead bodies may be aligned or not aligned. One possible advantage of the arrangements in FIGS. 11 and 12 is that, at least in some embodiments, the lead bodies can be percutaneously implanted individually using an introducer. Paddle leads are often surgically implanted.

Some of the components (for example, a power source 1212, an antenna 1218, a receiver 1202, and a processor 1204) of the electrical stimulation system can be positioned on one or more circuit boards or similar carriers within a sealed housing of an implantable pulse generator, if desired. Any power source 1212 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1218 or a secondary antenna. The external powder source can be in a device that is mounted on the skin of the user or in a unit that is provided near the user on a permanent or periodic basis.

If the power source 1212 is a rechargeable battery, the battery may be recharged using the optional antenna 1218, if desired. Power can be provided to the battery for recharging by inductively coupling the battery through the antenna to a recharging unit 1216 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 1204 is generally included to control the timing and electrical characteristics of the electrical stimulation system. For example, the processor 1204 can, if desired, control one or more of the timing, frequency, strength, duration, and waveform of the pulses. In addition, the processor 1204 can select which electrodes can be used to provide stimulation, if desired. In some embodiments, the processor 1204 selects which electrode(s) are cathodes and which electrode(s) are anodes. In some embodiments, the processor 1204 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1208 that, for example, allows modification of pulse characteristics. In the illustrated embodiment, the processor 1204 is coupled to a receiver 1202 which, in turn, is coupled to the optional antenna 1218. This allows the processor 1204 to receive instructions from an external source to, for example, direct the pulse characteristics and the selection of electrodes, if desired.

In one embodiment, the antenna 1218 is capable of receiving signals (e.g., RF signals) from an external telemetry unit 1206 which is programmed by the programming unit 1208. The programming unit 1208 can be external to, or part of, the telemetry unit 1206. The telemetry unit 1206 can be a device that is worn on the skin of the user or can be carried by the user and can have a form similar to a pager, cellular phone, or remote control, if desired. As another alternative, the telemetry unit 1206 may not be worn or carried by the user but may only be available at a home station or at a clinician's office. The programming unit 1208 can be any unit that can provide information, to the telemetry unit 1206 for transmission to the electrical stimulation system 1200. The programming unit 1208 can be part of the telemetry unit 1206 or can provide signals or information to the telemetry unit 1206 via a wireless or wired connection. One example of a suitable programming unit is a computer operated by the user or clinician to send signals to the telemetry unit 1206.

The signals sent to the processor 1204 via the antenna 1218 and the receiver 1202 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1200 to cease operation, to start operation, to start charging the battery, or to stop charging the battery. in other embodiments, the stimulation system does not include the antenna 1218 or receiver 1202 and the processor 1204 operates as programmed.

Optionally, the electrical stimulation system 1200 may include a transmitter (not shown) coupled to the processor 1204 and the antenna 1218 for transmitting signals back to the telemetry unit 1206 or another unit capable of receiving the signals. For example, the electrical stimulation system 1200 may transmit signals indicating whether the electrical stimulation system 1200 is operating properly or not or indicating when the battery needs to be charged or the level of charge remaining in the battery. The processor 1204 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

1. A spinal cord stimulation lead, comprising:
a paddle body configured and arranged to be implanted into the patient to stimulate a portion of the spinal cord of the patient, the paddle body having a distal end, a proximal end, and a longitudinal length extending from the distal end to the proximal end;
at least one lead body having a distal end portion, a proximal end portion, and a longitudinal length, the distal end portion of each of the at least one lead body being coupled to the proximal end of the paddle body;
a plurality of electrodes disposed on the paddle body, wherein the plurality of electrodes form at least two longitudinal columns including a first column and a second column, wherein the first and second columns are spaced apart laterally by at least 7 mm, center-to-center;
a plurality of terminals disposed along the proximal end portion of the at least one lead body; and
a plurality of conductors electrically coupling the plurality of terminals to the plurality of electrodes.

2. The spinal cord stimulation lead of claim 1, wherein the at least two longitudinal columns further include a third column disposed at least partially between the first and second columns.

3. The spinal cord stimulation lead of claim 2, wherein the at least two longitudinal columns further include a fourth column disposed at least partially between the first and second columns.

4. The spinal cord simulation lead of claim 3, wherein the first and second columns are configured and arranged for stimulation of dorsal horus of an average adult human when the paddle body is implanted along a midline of a spinal cord.

5. The spinal cord stimulation lead of claim 4, wherein the third and fourth columns are configured and arranged for stimulation of a dorsal column of an average adult human when the paddle body is implanted along a midline of a spinal cord.

6. The spinal cord stimulation lead of claim 2, wherein the third column includes more electrodes than the first column and more electrodes than the second column.

7. The spinal cord stimulation lead of claim 1, wherein the at least two longitudinal columns further include a third column disposed along a midline of the paddle body.

8. The spinal cord stimulation lead of claim 1, wherein the at least two longitudinal columns further include a third column and a fourth column disposed on the paddle body and spaced apart laterally by no more than 5 mm, center-to-center.

9. The spinal cord stimulation lead of claim 1, wherein the third and fourth columns do not longitudinally overlap with the first and second columns.

10. The spinal cord stimulation lead of claim 1, wherein the third and fourth columns are longitudinally separated from the first and second columns by at least 2 mm.

11. A spinal cord stimulation system, comprising:
the spinal cord stimulation lead of claim 1;
a control module coupleable to the spinal cord stimulation lead.

12. A method of spinal cord stimulation, the method comprising
implanting at least one electrical stimulation lead over a spinal cord of a patient so that a first column of electrodes is disposed over a first dorsal horn of the spinal cord and a second column of electrodes is disposed over a second dorsal horn of the spinal cord that is opposite the first dorsal horn;
coupling the at least one electrical stimulation lead to a control module; and
applying a stimulation current generated by the control module to at least one of the first or second dorsal horns using at least one of the electrodes of the first or second columns of electrodes.

13. The method of claim 12, wherein implanting at least one electrical stimulation lead comprises implanting a paddle lead, the paddle lead comprising a paddle body, wherein the first and second columns of electrodes are disposed on the paddle body.

14. The method of claim 13, wherein the first and second columns are spaced apart laterally by a distance, center-to-center, of at least 7 mm.

15. The method of claim 12, implanting at least one electrical stimulation lead comprises implanting a first cylindrical lead body having the first column of electrodes disposed thereon and a second cylindrical lead body having the second column of electrodes disposed thereon.

16. The method of claim 15, wherein the electrodes of the first column are ring electrodes.

17. The method of claim 15, wherein the electrodes of the first column are segmented electrodes, wherein each segmented electrode extends no more than 75% around a circumference of the first cylindrical lead body.

18. The method of claim 15, wherein the first lead body is part of a first lead and the second lead body is part of a second lead.

19. The method of claim 12, wherein the at least one electrical stimulation lead comprises at least one third column of electrodes, the method further comprising applying a stimulation current generated by the control module to a dorsal column of the patient using at leas t one of the electrodes of the at least one third column electrodes.

20. The method of claim 12, wherein applying a stimulation current comprises applying a stimulation current generated by the control module to both of the first and second dorsal horns using at least one of the electrodes of the first or second columns of electrodes.
